(19) United States
(12) Patent Application Publication
(10) Pub. No.: US 2024/0408140 A1
(43) Pub. Date: Dec. 12, 2024

(54) ENHANCING ANTICANCER FUNCTION OF NATURAL KILLER CELL THROUGH REGULATION OF ANTIOXIDANT MECHANISM

(71) Applicant: DAEGU GYEONGBUK INSTITUTE OF SCIENCE AND TECHNOLOGY, Daegu (KR)

(72) Inventors: Young Tae JEONG, Daegu (KR); Byungmoo OH, Daegu (KR)

(73) Assignee: DAEGU GYEONGBUK INSTITUTE OF SCIENCE AND TECHNOLOGY, Daegu (KR)

(21) Appl. No.: 18/704,511

(22) PCT Filed: Oct. 25, 2022
(86) PCT No.: PCT/KR2022/016315

§ 371 Date: Apr. 25, 2024

(30) Foreign Application Priority Data
Oct. 25, 2021 (KR) 10-2021-0142365
Oct. 21, 2022 (KR) 10-2022-0136591

(51) Int. Cl. A61K 35/17; A61P 35/00; C12N 5/0783

(52) U.S. Cl. CPC A61K 35/17; A61P 35/00; C12N 5/0646; C12N 2500/30; C12N 2500/32; C12N 2501/2302; C12N 2510/00

(57) ABSTRACT

The present invention relates to enhancing the anticancer function of a natural killer (NK) cell through the regulation of an antioxidant mechanism and confirms that, in comparison to a conventional NK cell, anticancer activity is enhanced in an NK cell having increased antioxidant activity due to the removal of the keap 1 gene or due to having been cultured in a medium containing an antioxidant material, and can thus be useful as a composition for preventing, treating, or alleviating cancer.

### TECHNICAL FIELD

The present disclosure relates to enhancement of an anticancer function of natural killer (NK) cells through regulation of an antioxidant mechanism.

### BACKGROUND ART

Natural killer (hereinafter, referred to as NK) cells are the main cells that constitutes the innate immune system and present in about 5˜20% of circulating lymphocytes. Normally, NK cells are non-specific to antigens that they recognize, encounter, and kill abnormal cells including tumor cells and virus-infected cells. A process by which NK cells kill tumor cells is carried out by selectively killing tumor cells by releasing cytoplasmic granules including granzyme B and perforin and secreting cytokines such as interferon-y and tumor necrosis factor-α (TNF-α). Owing to these characteristics of NK cells, the development of anticancer immune cell treatment using NK cells is actively underway to overcome the limitations of existing anticancer therapies.

However, in order to use NK cells as an effective anticancer immune cell treatment, it is necessary not only to secure a large number of NK cells, but also to activate NK cells more effectively. Since NK cells in a normal state are mostly in an inactivated state, it is essential to undergo a process of activating, in vitro, NK cells obtained from normal blood or inactivated blood of patients.

The Keap1-Nrf2 signaling mechanism is a representative antioxidant mechanism in our body that is responsible for regulation of production of reactive oxygen species (ROS). ROS is a major secondary signaling substance in cells, with reports that the Keap1-Nrf2 signaling mechanism that regulates ROS not only modulates the main inflammatory signaling mechanism, but also controls the differentiation and activation of various immune cells such as hematopoietic stem cells, B cells, and regulatory T cells.

### DISCLOSURE OF THE INVENTION

### Technical Goals

An object of the present disclosure is to provide an NK cell with a keap1 gene deleted.

Another object of the present disclosure is to provide a medium composition for NK cell activation, including an antioxidant as an active ingredient.

Another object of the present disclosure is to provide a pharmaceutical composition for preventing or treating cancer diseases, including the keap1 gene-deleted NK cell or an NK cell cultured with the medium composition as an active ingredient.

Another object of the present disclosure is to provide a health functional food composition for preventing or ameliorating cancer diseases, including the keap1 gene-deleted NK cell or an NK cell cultured with the medium composition as an active ingredient.

### Technical Solutions

To achieve the above objects, the present disclosure provides an NK cell with a keap1 gene deleted.

In addition, the present disclosure provides a medium composition for NK cell activation, including an antioxidant as an active ingredient.

In addition, the present disclosure provides a pharmaceutical composition for preventing or treating cancer diseases, including the keap1 gene-deleted NK cell or an NK cell cultured with the medium composition as an active ingredient.

In addition, the present disclosure provides a health functional food composition for preventing or ameliorating cancer diseases, including the keap1 gene-deleted NK cell or an NK cell cultured with the medium composition as an active ingredient.

### Advantageous Effects

According to the present disclosure, it is identified that an NK cell with a keap1 gene deleted or cultured in a medium containing an antioxidant to promote antioxidant activity exhibits enhanced anticancer activity compared to a conventional NK cell, and thus the NK cells may be useful as a composition for preventing, treating, or ameliorating cancer diseases.

### BRIEF DESCRIPTION OF DRAWINGS

FIG. 1 shows an overview of a system in which an anticancer function of NK cells is enhanced by deletion of a keap1 gene or antioxidant treatment.

FIG. 2 shows a result of analyzing changes in expression of a keap1 gene and target genes of nrf2 (GCLC and G6PD2) in keap1 gene-deleted NK cells.

FIG. 3 shows results of analyzing (A) changes in maturity of keap1 gene-deleted NK cells and (B) cytotoxicity against cancer cells in the co-culture with cancer cells.

FIG. 4 shows results of analyzing the cytotoxicity against cancer cells in the co-culture of keap1 gene-deleted NK cells and lung cancer organoids.

FIG. 5 shows results of analyzing the incidence of skin cancer when the de novo development of skin cancer is induced in mice with a keap1 gene deleted from NK cells.

FIG. 6 shows results of analyzing the cytotoxicity against cancer cells in the co-culture of cancer cells with NK cells cultured in an antioxidant-including medium. NAC represents NK cells cultured in an antioxidant medium treated with N-acetylcysteine, Tempol represents NK cells cultured in an antioxidant medium treated with tempol, and Control represents normal NK cells.

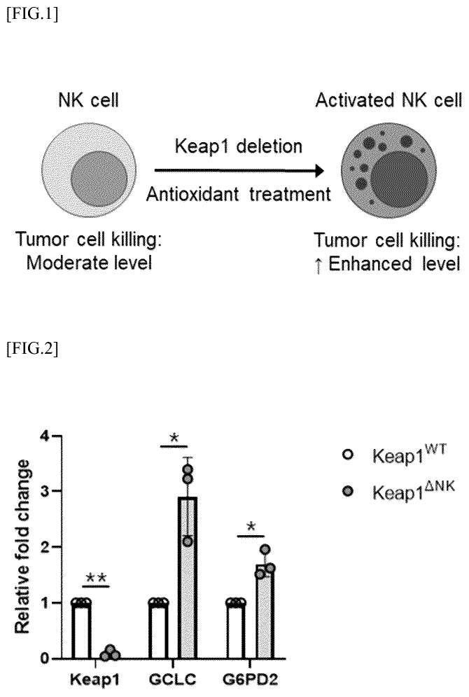

### BEST MODE FOR CARRYING OUT THE INVENTION

Hereinafter, the present disclosure will be described in detail.

The present disclosure provides an NK cell with a keap1 gene deleted.

The NK cell may have decreased expression of the keap1 gene and increased expression of a GCLC or G6PD2 gene.

In addition, the NK cell may suppress over-maturation of cells.

In addition, the NK cell may have increased anticancer activity.

The term “NK cell” as used herein may be, but is not limited to, one or more selected from the group consisting of immortalized NK cell lines, hematopoietic stem cell-derived NK cells, peripheral blood-derived NK cells, NK cells induced by reprogramming, and animal-derived NK cells.

In addition, the present disclosure provides a medium composition for NK cell activation, including an antioxidant as an active ingredient.

The antioxidant may be, but is not limited to, one or more selected from the group consisting of N-acetylcysteine, 4-hydroxy-2,2,6,6-tetramethylpiperidine 1-oxyl (TEMPOL), vitamin A, vitamin C, vitamin E, and β-carotene.

The medium composition may further include, but not limited to, one or more selected from the group consisting of cytokines, L-glutamine, HEPES, sodium pyruvate, 2-mercaptoethanol, fetal bovine serum (FBS), and antibiotic-antifungal agents.

The medium may be, but is not limited to, one selected from the group consisting of Roswell Park Memorial Institute Medium 1640 (RPMI1640), Dulbecco's Modified Eagle's Medium (DMEM), Minimal essential Medium (MEM), Basal Medium Eagle (BME), F-10, F-12, α-Minimal essential Medium (α-MEM), Glasgow's Minimal essential Medium (GMEM), Iscove's Modified Dulbecco's Medium (IMDM), DMEM/F12, and Advanced DMEM/F12.

The antibiotic-antifungal agents may be, but are not limited to, two or more selected from the group consisting of penicillin, streptomycin, gentamicin, nystatin, and amphotericin.

The term “cytokine” as used herein refers to a variety of small-sized proteins that are produced by various cells and used in signal transduction capable of regulating the function of other cells, including their own, further including, but not limited to, interleukin-2 (IL-2), IL-3, IL-5, IL-6, IL-7, IL-15, IL-21, or a combination thereof.

An “antioxidant” as used herein refers to a substance that is introduced into cells to remove ROS in cells, and the term “activation” as used herein refers to enhancing of the cytotoxicity of NK cells.

The term “medium” as used herein refers to a culture solution that includes components necessary for the growth, survival, or differentiation of NK cells in vitro while enabling the growth, survival, or differentiation, and includes all conventional media appropriate for culture and differentiation used in the field of immunology. Depending on the type of cell or the technical level of the field, there may be variations given to the type of medium and the culture condition. The medium used for NK cell culture may generally be a Minimum Essential Medium including carbon sources, nitrogen sources, and trace element components.

In addition, the present disclosure provides an NK cell cultured with the medium composition.

In addition, the present disclosure provides a pharmaceutical composition for preventing or treating cancer diseases, including the keap1 gene-deleted NK cell or the NK cell cultured with the medium composition as an active ingredient.

The cancer may be, but is not limited to, one or more selected from the group consisting of breast cancer, cervical cancer, glioma, brain cancer, melanoma, lung cancer, bladder cancer, prostate cancer, leukemia, kidney cancer, liver cancer, colorectal cancer, pancreatic cancer, stomach cancer, gallbladder cancer, ovarian cancer, lymphoma, osteosarcoma, uterine cancer, oral cancer, bronchial cancer, nasopharyngeal cancer, laryngeal cancer, skin cancer, blood cancer, thyroid cancer, parathyroid cancer, and ureteral cancer.

The pharmaceutical composition of the present disclosure may be prepared in a unit dose form or prepared by infusion in a multi-dose container through formulation using pharmaceutically acceptable carriers according to a method that may be easily carried out by a person skilled in the art to which the present disclosure pertains.

The pharmaceutically acceptable carriers are those commonly used in preparation, including, but not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present disclosure may further include lubricants, wetting agents, sweetening agents, flavoring agents, emulsifying agents, suspending agents, and preservatives, in addition to the above components.

In the present disclosure, the content of additives included in the pharmaceutical composition is not particularly limited and may be appropriately adjusted within the content range used for conventional preparation.

The pharmaceutical composition may be formulated, but is not limited to, in the

form of one or more external skin preparations selected from the group consisting of injectable formulations such as aqueous solutions, suspensions, and emulsions, pills, capsules, granules, tablets, creams, gels, patches, sprays, ointments, emplastrum agents, lotions, liniments, pastas, and cataplasmas.

The pharmaceutical composition of the present disclosure may include a pharmaceutically acceptable carrier and diluent, which are additional for the formulation. The pharmaceutically acceptable carrier and diluent include, but are not limited to, excipients such as starch, sugar, and mannitol, fillers and extenders such as calcium phosphate, cellulose derivatives such as carboxymethylcellulose and hydroxypropyl cellulose, binders such as gelatin, alginate, and polyvinylpyrrolidone, lubricants such as talc, calcium stearate, hydrogenated castor oil, and polyethylene glycol, disintegrants such as povidone and crospovidone, and surfactants such as polysorbates, cetyl alcohol, and glycerol. The pharmaceutically acceptable carrier and diluent may be biologically and physiologically compatible with subjects. Examples of the diluent may include, but are not limited to, saline, aqueous buffers, solvents, and/or dispersion media.

The pharmaceutical composition of the present disclosure may be administered orally or parenterally (e.g., intravenously, subcutaneously, intraperitoneally, or topically) depending on a desired method. For oral administration, the pharmaceutical composition may be formulated as tablets, troches, lozenges, aqueous suspensions, oily suspensions, powder preparation, granules, emulsions, hard capsules, soft capsules, syrups, or elixirs. For parenteral administration, the pharmaceutical composition may be formulated as injections, suppository agents, powder for respiratory inhalation, aerosols for sprays, ointments, powder for application, oil, and creams.

The dosage range of the pharmaceutical composition of the present disclosure may vary depending on the patient's condition, body weight, age, sex, health status, dietary constitution specificity, the nature of preparations, the degree of diseases, administration duration of the composition, administration methods, administration periods or intervals, excretion rate, and drug forms, and be appropriately selected by those skilled in the art. For example, the dosage may be in the range of about 0.1 to 10,000 mg/kg but is not limited thereto, while it may be administrated in divided doses from one to several times a day.

The pharmaceutical composition may be administered orally or parenterally (e.g., intravenously, subcutaneously, intraperitoneally, or topically) depending on a desired method. A pharmaceutically effective amount and effective dosage of the pharmaceutical composition of the present disclosure may vary depending on preparation methods, administration methods, administration duration, and administration routes of the pharmaceutical composition, and those skilled in the art may easily determine and prescribe the dosage that is effective for desired treatment. Administration of the pharmaceutical composition of the present disclosure may be conducted once a day or several times in divided doses.

In addition, the present disclosure provides a health functional food composition for preventing or ameliorating cancer diseases, including the keap1 gene-deleted NK cell or the NK cell cultured with the medium composition as an active ingredient.

The present disclosure may be generally used as a commonly used food product.

The food composition of the present disclosure may be used as a health functional food. The term “health functional food” as used herein refers to food manufactured and processed with raw materials or ingredients having useful functionality for the human body in accordance with the Health Functional Food Act, and the term “functionality” as used herein refers to the intake to derive effectiveness in health care such as physiological actions or regulation of nutrients for the structure and function of the human body.

The health functional food composition may include conventional food additives, and the suitability as the “food additive” is determined by the standards and criteria related to corresponding items according to the general rules and general test methods of Korean Food Additives Codex approved by the Ministry of Food and Drug Safety, unless otherwise stipulated.

The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

The food composition of the present disclosure may be manufactured and processed in the form of tablets, capsules, powder, granules, liquids, and pills. For example, hard capsule preparations among health functional foods in the form of capsules may be prepared by mixing and filling the composition according to the present disclosure in conventional hard capsules along with additives such as excipients, and the soft capsule preparations may be manufactured by mixing the composition according to the present disclosure with the additives such as excipients and then filling the same in capsule bases such as gelatin. The soft capsule preparations may include, if necessary, plasticizers such as glycerin or sorbitol, colorants, and preservatives.

The definition of terms for the excipient, binder, disintegrant, lubricant, flavor enhancer, and flavoring agent is described in documents known in the art and includes those having the same or similar functions. The type of food is not particularly limited and includes all health functional foods in the ordinary sense.

The term “prevention” as used herein refers to any action of suppressing or delaying cancer diseases by administering the composition according to the present disclosure.

The term “treatment” as used herein refers to any action that ameliorates or favorably changes the cancer diseases by administering the composition according to the present disclosure.

The term “amelioration” as used herein refers to any action of ameliorating the bad condition of cancer diseases by administering the composition according to the present disclosure.

### Modes for Carrying Out the Invention

Hereinafter, to help understanding of the present disclosure, example embodiments will be described in detail. However, the following example embodiments are merely illustrative of the content of the present disclosure, and the scope of the present disclosure is not limited to the following example embodiments. The example embodiments of the present disclosure are provided to more completely explain the present disclosure to those skilled in the art.

### [Experiment Example 1] Isolation of keap1 Gene-Deleted NK Cells

1-1. Preparation of keap1 Gene-Deleted Mice

Animal experiments related to this patent were conducted with the approval of the Institutional Animal Care and Use Committee in Institute of Science and Technology, Daegu, Gyeongbuk (Approval number: DGIST-IACUC-22100612-0002). To delete the keap 1gene from NK cells isolated from mice, NCR^{icre} mice expressing cre recombinase in NK cells was crossed with keap 1^{flox/flox} mice to prepare keap 1^{flox/flox};Ncr1^{icre} (hereinafter referred to as keap 1^{ΔNK}) mice in which expression of the keap1 gene is reduced specifically in NK cells. As a control, NCR^{icre} mice and keap 1^{wt} mice were crossed to prepare keap1^{wt};Ncr1^{icre} (hereinafter referred to as keap1^{WT}) mice normally expressing the keap1 gene.

### 1-2. Isolation of Blood Cells From Mouse Spleen

The spleen of keap 1^{WT} and keap1^{ΔNK} mice was extracted, washed thoroughly with phosphate buffered saline (hereinafter referred to as PBS), and then finely ground using a 40 μm cell strainer (SPL, #93040) in 5 ml of RPMI1640. Finely ground spleen was collected in a 50 ml conical tube, and then 20 ml of PBS, in which 2% fetal bovine serum (hereinafter referred to as FBS) is included, was added, followed by centrifugation for 5 minutes and removal of the supernatant. Centrifuged spleen cells were added with 1× red blood cell (RBC) lysis buffer to release thoroughly by pipetting, and cultured for 5 minutes in a cell culture machine supplied with 5% carbon dioxide at 37° C. Spleen cells with red blood cells dissociated were centrifuged for 5 minutes by adding 20 ml of PBS including 2% FBS, and then the supernatant was removed. Centrifuged spleen cells were suspended using RPMI1640 medium, and cell count was measured.

### 1-3. Isolation of NK Cells

The spleen cells extracted from the Experimental Example 1-1 were isolated from the NK cells using the FACSAriaTMIII cell sorter (BD bioxciences). Specifically, the extracted spleen cells were treated with antibodies for CD3 as a T cell marker, CD19 as a B cell marker, NK1.1 and CD49b as NK cell markers and then reacted at 4° C. for 30 minutes, and the CD3-negative, CD19-negative, NK1.1-positive, and CD49b-positive parts were separated using a FACSAriaTMIII cell sorter.

### [Experimental Example 2] Preparation of NK Cell Activation Media Including Antioxidants

NK cell activation media were prepared as follows. In an NK cell culture medium obtained by mixing 10% FBS, 1× penicillin/streptomycin, 1× glutamax, 1× HEPES, 0.5 mM sodium pyruvate, 50 μM 2-mercaptoethanol, and 50 ng/ml interleukin-2 (IL-2) in RPMI1640 medium, 1 mM N-acetylcysteine or 0.5 mM 4-hydroxy-2,2,6,6-tetramethylpiperidine 1-oxyl (TEMPOL) was blended.

### [Example 1] Analysis on Expression of keap1 Genes and nrf2 Target Genes

In order to identify the decrease in keap1 gene expression in keap1 gene-deleted NK cells, analyzed was the expression of the keap1 gene as well as GCLC and G6PD2, which are target genes of nrf2 and transcription factors that the protein stability increases in accordance with a decrease in the expression of the keap1 gene. The NK cells were subjected to extraction of mRNA using an RNA purification kit (Monarch total RNA miniprep kit, NEB, #T2010), and cDNA was synthesized using a cDNA synthesis kit (High-capacity cDNA reverse transcription kit, Thermo, #4368814). Afterwards, quantitative polymerase chain reaction (q-PCR) was performed with the synthesized cDNA as the template to analyze the expression of keap1, GCLC, and G6PD2 genes.

As a result, as shown in FIG. 2, the expression of keap1 gene decreased in keap1 gene-deleted NK cells, while that of the GCLC and G6PD2 genes increased.

### Example 2: Analysis on the Cell Maturity

NK cells differentiate and mature from hematopoietic stem cells, which allows distinction of the maturity according to the pattern of markers that are specifically expressed in each stage. Thereamong, the maturity of NK cells in the final three stages may be distinguished in the order of 1) CD27 positive and CD11b negative; 2) CD27 positive and CD11b positive; and 3) CD27 negative and CD11b positive.

Therefore, to determine the maturity of keap1 gene-deleted NK cells, FACS analysis was performed using CD27 and CD11b antibodies.

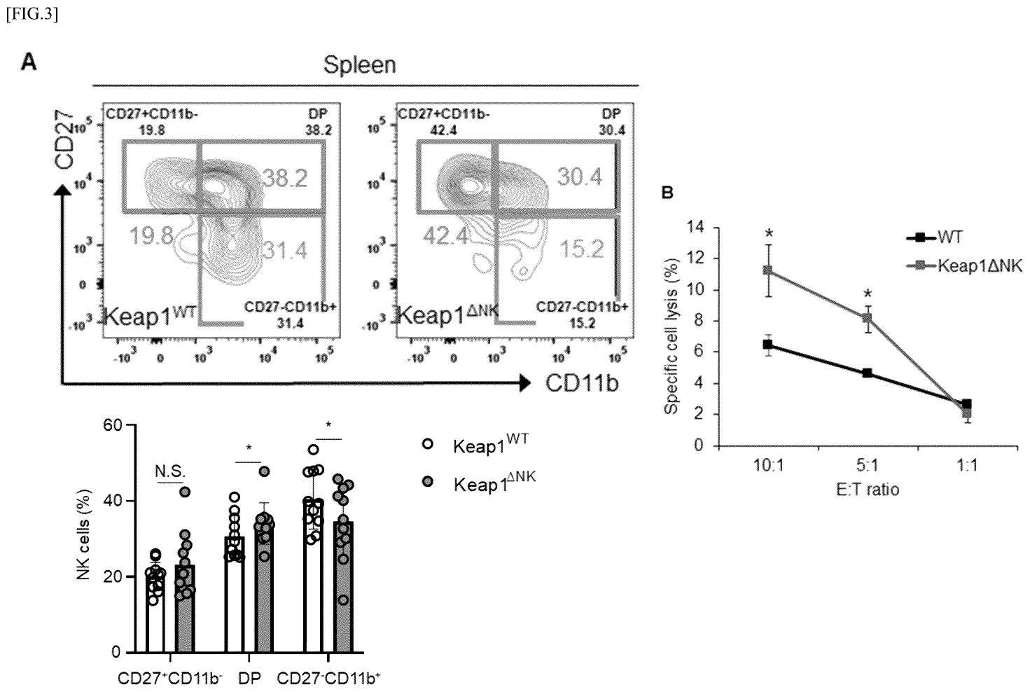

As a result, as shown in FIG. 3A, it was found that over-maturation was suppressed in keap1 gene-deleted NK cells.

### [Example 3] Analysis on the Cytotoxicity Against Cancer Cells

### 3-1. Cytotoxicity Analysis via Co-Culture of NK Cells and Yac-1 Cells

In order to identify the cytotoxicity of keap1 gene-deleted NK cells against cancer cells, the NK cells were co-cultured with the Yac-1 cell line, which is a mouse lymphoma cell. The Yac-1 cell line was added in RPMI1640 (10% FBS/RPMI1640) medium including 10% FBS, treated with 5 μg/ml Calcein AM (Invitrogen, C1430), and cultured for 1 hour in a cell culture machine at 37° C. in the presence of 5% carbon dioxide conditions to label with fluorescence. Afterwards, the NK cells and fluorescence-labeled Yac-1 cells were mixed in a ratio of 10:1, 5:1, and 1:1 and dispensed in a round bottom 96-well plate (SPL, #34096). Additionally, fluorescence-labeled Yac-1 cells were also dispensed into the wells of the maximum (Maximum, 10% FBS/RPMI1640 including 2% Triton X-100) and minimum (Minimum, 10% FBS/RPMI1640) to calculate the cytotoxicity rate. After dispensing each cell, the cells were cultured for 4 hours in a cell culture machine at 37° C. in the presence of 5% carbon dioxide. After culture for 4 hours, the medium was mixed well and centrifuged at 100×g for 5 minutes, the supernatant was transferred to a black plate (SPL, #30296), and fluorescence was measured using a fluorescence microplate reader. Fluorescence was measured at an excitation wavelength of 485 nm and an emission wavelength of 530 nm, and cytotoxicity was calculated using the following Equation 1.

Cytotoxicity=[(Test fluorescence−minimum fluorescence)/(maximum fluorescence−minimum fluorescence)]×100 [Equation 1]

As a result, as shown in FIG. 3B, the cytotoxicity of keap1-deleted NK cells was higher than that of normal NK cells.

### 3-2. Cytotoxicity Analysis via Co-Culture of NK Cells and Mouse Lung Cancer Organoids

In order to determine the cytotoxicity of keap1 gene-deleted NK cells against cancer cells, the NK cells were co-cultured with mouse (C57b6/B129) lung cancer organoids, cultured in a 24-well plate with the p53 gene and keap1 gene deleted. After collecting mouse lung cancer organoids cultured in size of 50-100 μm from 24-well plates, some thereof were enzymatically dissociated using trypsin, followed by the cell count measurement. The cell-counted lung cancer organoids were mixed with lung cancer organoid culture medium and matrigel (Corning, #356231) in a ratio of 1:1 and dispensed in a well which is hardened after putting in 96-well plate in advance, followed by culture in a cell culture machine at 37° C. in the presence of 5% carbon dioxide for 30-60 minutes. Afterwards, the NK cells were dispensed in a ratio of 5:1 and cultured for 24 hours in a cell culture machine at 37° C. in the presence of 5% carbon dioxide. Co-cultured wells were enzymatically dissociated from matrigel using PBS including 1× dispase (Stem cell, #07913). Thereafter, it was added into 0.25% trypsin-EDTA (0.25% Trypsin-EDTA; Gibco, #25200-072) solution and subjected to enzymatic dissociation for 5 minutes in a cell culture machine at 37° C. in the presence of 5% carbon dioxide. After thoroughly releasing enzymatically dissociated lung cancer organoids by pipetting, PBS including 2% FBS was added to neutralize 0.25% trypsin/0.05% EDTA, and then centrifugation was performed for 5 minutes, followed by removal of the supernatant. Centrifuged lung cancer organoids were stained using the Zombie NIRTM fixable viability kit (Biolegend, #423105), and the proportion of cells killed by FACS was analyzed.

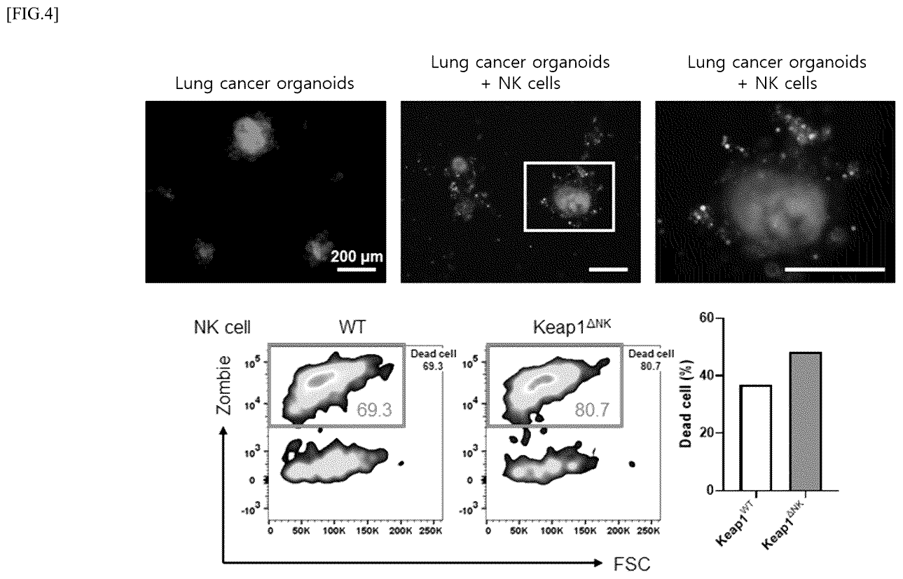

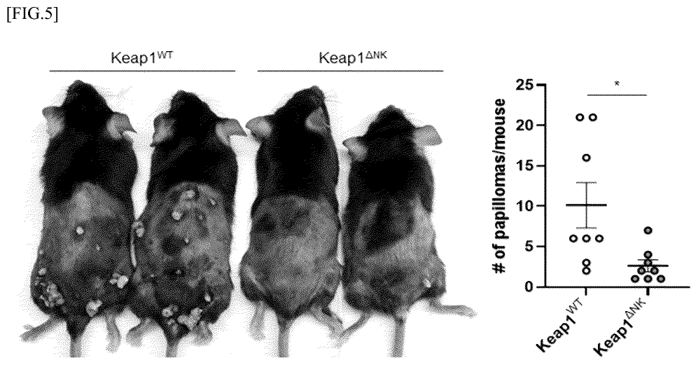

As a result, as shown in FIG. 4, it was found that the cytotoxicity of keap1-deleted NK cells was higher than that of normal NK cells.

### [Example 4] Analysis on the Anticancer Activity

In order to identify the anticancer activity on skin cancer caused by de novo of the keap1 gene-deleted NK cells, hair on the back of keap1^{ΔNK} and keap1^{WT} mice were eliminated and sprayed with 200 μl of 0.25 mg/ml 7,12-dimethylbenz[a]anthracene (DMBA) which is dissolved in acetone. Afterwards, 200 μl of 0.02 mg/ml phorbol 12-myristate 13-acetate (TPA) dissolved in acetone was sprayed once a week for 30-40 weeks to check the development patterns of skin cancer.

As a result, as shown in FIG. 5, it was observed that, in the case of mice with the keap1 gene deleted from NK cells, the incidence of skin cancer decreased. In conclusion, it was found that the de novo production of cancer was inhibited when enhancing antioxidant activity by removing the keap1 gene specifically for NK cells.

### [Example 5] Analysis on the Anticancer Activity of NK Cells Cultured in NK Cell Activation Medium Including Antioxidants

In order to determine whether NK cells cultured in NK cell activation medium including antioxidants may show the same anticancer activity as the keap1 gene-deleted NK cells, NK cells isolated from the mouse spleen were placed in the NK cell activation medium prepared according to the Experimental Example 2 and cultured in a cell culture machine at 37° C. in the presence of 5% carbon dioxide for 30-60 minutes. Afterwards, the cytotoxicity was checked in the same manner as in Example 3-1.

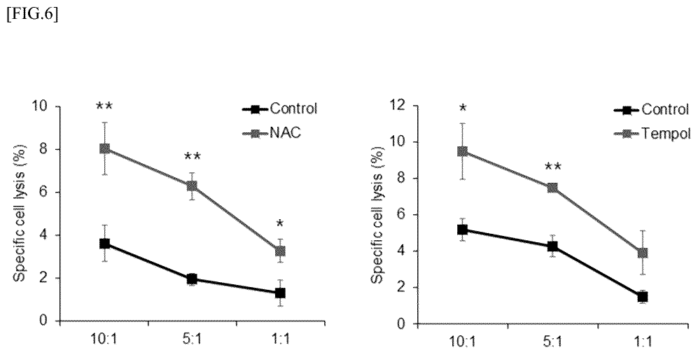

As a result, as shown in FIG. 6, both NK cells (NAC) cultured in an antioxidant medium treated with N-acetylcysteine and NK cells (Tempol) cultured in an antioxidant medium treated with Tempol were found to have higher cytotoxicity than normal NK cells (control). In conclusion, it was found that normal NK cells could also exhibit similar anticancer activity as the keap1 gene-deleted NK cells by culturing in the NK cell activation medium including antioxidants.

As described above, a specific part of the content of the present disclosure is described in detail, for those skilled in the art, it is clear that the specific description is merely a preferred example embodiment, and the scope of the present disclosure is not limited thereby. In other words, the substantial scope of the present disclosure is defined by the appended claims and their equivalents.

1. A natural killer (NK) cell with a keap1 gene deleted.

2. The NK cell of claim 1, wherein the NK cell has decreased expression of the keap1 gene and increased expression of a GCLC or G6PD2 gene.

3. The NK cell of claim 1, wherein the NK cell suppresses over-maturation of cells.

4. The NK cell of claim 1, wherein the NK cell has increased anticancer activity.

5. A method of enhancing an anticancer activity in natural killer (NK) cells, comprising:
isolating the NK cells from a subject;
adding the isolated NK cells in a medium composition comprising an antioxidant as an active ingredient; and
culturing the NK cells in the medium composition to activate the NK cells.

6. The method of claim 5, wherein the antioxidant is one or more selected from the group consisting of N-acetylcysteine, 4-hydroxy-2,2,6,6-tetramethylpiperidine 1-oxyl (TEMPOL), vitamin A, vitamin C, vitamin E, and β-carotene.

7. The method of claim 5, wherein the medium composition further comprises one or more selected from the group consisting of cytokines, L-glutamine, HEPES, sodium pyruvate, 2-mercaptoethanol, fetal bovine serum (FBS), and antibiotic-antifungal agents.

8. The method of claim 5, wherein the medium is one selected from the group consisting of Roswell Park Memorial Institute Medium 1640 (RPMI1640), Dulbecco's Modified Eagle's Medium (DMEM), Minimal essential Medium (MEM), Basal Medium Eagle (BME), F-10, F-12, α-Minimal essential Medium (α-MEM), Glasgow's Minimal essential Medium (GMEM), Iscove's Modified Dulbecco's Medium (IMDM), DMEM/F12, and Advanced DMEM/F12.

9. The method of claim 7, wherein the antibiotic-antifungal agents are two or more selected from the group consisting of penicillin, streptomycin, gentamicin, nystatin, and amphotericin.

10. A method of preventing or treating cancer diseases, comprising:
administering a pharmaceutical composition comprising the NK cell of claim 1, as an active ingredient to a subject.

11. The method of claim 10, wherein the cancer is one or more selected from the group consisting of breast cancer, cervical cancer, glioma, brain cancer, melanoma, lung cancer, bladder cancer, prostate cancer, leukemia, kidney cancer, liver cancer, colorectal cancer, pancreatic cancer, stomach cancer, gallbladder cancer, ovarian cancer, lymphoma, osteosarcoma, uterine cancer, oral cancer, bronchial cancer, nasopharyngeal cancer, laryngeal cancer, skin cancer, blood cancer, thyroid cancer, parathyroid cancer, and ureteral cancer.

12. A method of preventing or ameliorating cancer diseases, comprising:
administering a health functional food composition comprising the NK cell of claim 1, as an active ingredient to a subject.

13. The method of claim 10, wherein the pharmaceutical composition comprises an NK cell cultured with a medium composition comprising an antioxidant as an active ingredient to the subject.

14. The method of claim 13, wherein the antioxidant is one or more selected from the group consisting of N-acetylcysteine, 4-hydroxy-2,2,6,6-tetramethylpiperidine 1-oxyl (TEMPOL), vitamin A, vitamin C, vitamin E, and β-carotene.

15. The method of claim 12, wherein the health functional food composition comprises an NK cell cultured with a medium composition comprising an antioxidant as an active ingredient to the subject.

16. The method of claim 15, wherein the antioxidant is one or more selected from the group consisting of N-acetylcysteine, 4-hydroxy-2,2,6,6-tetramethylpiperidine 1-oxyl (TEMPOL), vitamin A, vitamin C, vitamin E, and β-carotene.
